(19) United States
(12) Patent Application Publication
(10) Pub. No.: US 2005/0002947 A1
(43) Pub. Date: Jan. 6, 2005

(54) In vitro bioassay employing laboratory-cultured larvae of strongyloides stercoralis

(75) Inventors: Ralph D. Robinson, Kingston (JM); Lawrence A.D. Williams, Kingston (JM); John F. Lindo, Kingston (JM); Wayne M. Forbes, Butler, PA (US)

Correspondence Address: MILES & STOCKBRIDGE PC, 1751 PINNACLE DRIVE, SUITE 500, MCLEAN, VA, 22102-3833, US

(73) Assignee: University of the West Indies

(21) Appl. No.: 10/833,013

(22) Filed: Apr. 28, 2004

Related U.S. Application Data
(60) Provisional application No. 60/466,054, filed on Apr. 29, 2003.

(51) Int. Cl. A61K 39/00; A61K 39/38; A61K 31/415; A01N 43/56

(52) U.S. Cl. 424/184.1

(57) ABSTRACT

An in vitro bioassay method is provided by maintaining an infection of Strongyloides stercoralis in a host animal, multiplying excreted larvae in coproculture, and exposing the larvae suspended in liquid medium to materials capable of having a biological effect on the larvae. The assay method is useful in assaying the effect of candidate anthelmintic materials.

### RELATIONSHIP TO OTHER APPLICATIONS

This application claims the benefit of the priority of U.S. Provisional Patent Application No. 60/466,054, filed Apr. 29, 2003, the entire disclosure of which is incorporated herein by reference.

### BACKGROUND OF THE INVENTION

1. Field of the Invention

This invention relates to bioassays and more particularly to bioassays using in vitro cultured larvae of Strongyloides stercoralis.

2. Background of the Invention

Parasitic nematodes have long presented significant public health problems. Although some pharmaceutical compounds have been developed for treatment of nematode infections, e.g., albendazole, thiabendazole, and ivermectin, the search for more effective and safer anthelmintic medicaments continues to be of great importance. However, progress in screening and testing candidate anthelmintic compounds has been impeded by the lack of a convenient and economical in vitro method that might obviate animal testing at least in the early screening stage of the investigation. The problem is particularly acute for screening and testing of extracts of medical plants for anthelmintic activity.

Although larvae of S. stercoralis have been multiplied in culture in order to enhance the sensitivity of diagnostic testing for S. Stercoralis infections, such cultures have not been used for testing of biological effects of materials on such nematodes.

Accordingly, a need has continued to exist for an in vitro bioassay suitable for use in investigating biological activity of materials and, in particular, in preliminary screening and testing of substances for anthelmintic activity.

### SUMMARY OF THE INVENTION

According to the invention the parasitic nematode Strongyloides stercoralis is maintained in a host animal, excreted larvae are multiplied in in vitro culture, the larvae so obtained are contacted with materials that might exert a physiologic effect on the larvae, and the effect of such materials on the larvae is evaluated. Typically, the materials to be evaluated are candidate anthelmintic compounds, which are evaluated for their effectiveness in immobilizing or killing the larvae.

Accordingly, it is an object of the invention to provide an in vitro bioassay for evaluating biological activity of selected materials.

A further object is to provide a bioassay for evaluating the efficacy of candidate anthelmintic compounds.

A further object is to provide a bioassay suitable for screening and testing extracts of medicinal plants for anthelmintic activity.

Further objects of the invention will be apparent from the description which follows.

### BRIEF DESCRIPTION OF THE DRAWINGS

FIG. 1 is a photomicrograph of a stained culture of larvae of S. strongyloides showing dead (darkly stained) and living (unstained) larvae.

FIG. 2 shows a comparison of slopes relating to cumulative percentage mortality of S. stercoralis larvae in aqueous solutions containing methanol.

FIG. 3A shows the cumulative corrected percentage immobility of S. stercoralis larvae produced by different concentrations of Triton® X-100.

FIG. 3B shows the cumulative corrected percentage mortality of S. stercoralis larvae produced by different concentrations of Triton® X-100.

FIG. 4 shows a flow chart for a typical bioassay of the invention.

### DETAILED DESCRIPTION OF THE INVENTION AND PREFERRED EMBODIMENTS

The invention will be described in connection with a preferred embodiment thereof using larvae of Strongyloides stercoralis as a test organism.

Strongyloides stercoralis is a zoonotic, intestinal roundworm infecting some 100 million persons worldwide. Notwithstanding its significant public health importance, the organism presents a unique opportunity for investigators involved in screening novel compounds for biological activity. The possession of a heterogonic life cycle, whereby free-living adult worms produce large numbers of infective larvae in the external environment, lends this helminth to laboratory culture. The inventors have used this system safely and successfully in Jamaica to routinely screen and further study extracts of medicinal plants for anthelmintic activity.

Maintenance of S. stercoralis in Mongrel Dogs:

Two weaned mongrel puppies are housed in a restricted access, purpose-built kennel which allows for adequate treatment (bleaching) and disposal of faeces. The animals are treated for ecto- and endoparasites using ethically prescribed chemotherapeutics. Weekly faecal samples are examined for helminth infections; when four consecutive negative findings occur, the animals may be considered helminth-free and ready for inoculation. Although dogs are a preferred host for maintaining an infection of S. stercoralis, because of their ready availability and ease of maintenance, any mammal susceptible to infection with S. stercoralis, e.g., cats or higher primates, may be used as a host.

Filariform larvae are harvested from agar cultures of faeces of an infected human volunteer. Use of a single individual reduces potential genetic heterogeneity of cultured worm populations.

Larvae are maintained in Locke's nematode saline solution (LNS) containing 400 i.u./mL benzyl penicillin and 400 i.u./mL streptomycin sulfate. Each dog is inoculated subcutaneously in the scruff area with 1 mL LNS containing about 3000 larvae. Daily oral administration of prednisolone (0.5 mg/kg) is required to maintain the infection. The prepatent period in mongrel dogs is 7-10 weeks.

Laboratory Culture of S. stercoralis for Bioassay Work:

The infected host animals shed larvae in their faeces, and these larvae are harvested and multiplied by coproculture for use in the bioassay of the invention.

Coproculture of about 30 g of fresh, moistened canine faeces is conducted using granular charcoal (1:1 v:v) in sealed 10 cm Petri dishes. Cultures are maintained at 26° C. under semi-lighted conditions. S. stercoralis filariform larvae typically migrate to the surface of the culture and eventually into water droplets that condense on the inner surface of the lid. Larvae appearing within 2 days are mostly those accruing from the homogonic cycle and usually occur in insufficient numbers for bioassay. After five to eight days have elapsed, free living adult worms effect ˜50-fold increase in the density of infective larvae (heterogonic cycle).

Although other methods of culture, e.g., culture in agar containing appropriate nutrients for the adult worms, are possible, coproculture in sealed petri dishes as described above is preferred for its convenience. The temperature of the culture should preferably maintained at about 26° C.±4° C. The cultures should preferably be kept in subdued light to avoid the deleterious effects of full daylight on the larvae.

Results of experiments conducted in accordance with the procedure described above are presented in the following discussion.

Assessment of Larval Mortality:

Dye exclusion tests have been used successfully to indicate viability in parasitic platyhelminths, and similar testing can be applied to S. stercoralis larvae. Use of 0.01% v/v eosin in LNS (ELNS) in a dye exclusion test indicated death of larvae in less than 10 minutes, and 0.01-0.03% ELNS had no effect on healthy individuals. The larvae typically survive for more than 100 hours in ELNS. Accordingly, LNS is a preferred medium in which to maintain larvae harvested from multiplication in coproculture and ELNS is a preferred medium for evaluating the effects of physiologically active materials, such as candidate anthelmintic materials, on the larvae. However, the skilled practitioner will recognize that any medium in which the larvae can be maintained for a convenient period of time, and any appropriate vital staining dye, can be used in the method of the invention.

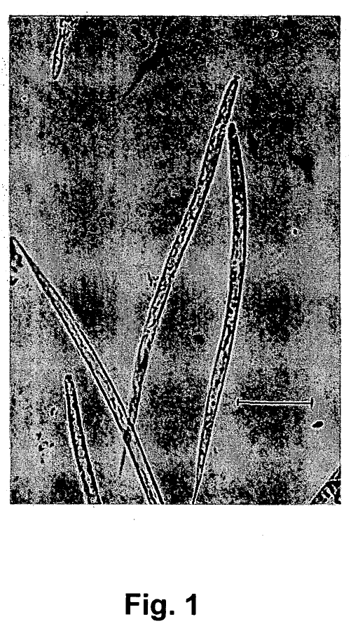

The results of a comparative test to establish the ability of a dye exclusion procedure using eosin to discriminate between live and killed larvae are presented in Table 1 below. FIG. 1 is a photomicrograph of a microscopic inspection field showing a number of larvae of S. stercoralis after exposure to an anthelmintic agent. The unstained (light colored) larvae are living, while the dark colored larvae have absorbed the staining dye, indicating that they have been killed by the active agent.

TABLE 1
Percentage (n˜100) of S. stercoralis larvae stained using
eosin in vitro

| | | Percentage stained with time | | | | | | | |
|---|---|---|---|---|---|---|---|---|---|
| Infective larvae | Eosin concentration (w/v) | 0 | 10 | 20 | 30 | 40 | 50 | 60 | (min) |
| Heat killed larvae (60° C./30 min) | 0.01% | 0 | 100 | | | | | | |
| | 0.03% | 0 | 100 | | | | | | |
| Living larvae | 0.01% | 0 | 1 | 1 | 2 | 2 | 2 | 2 | 2 |
| | 0.03% | 0 | 1 | 1 | 2 | 2 | 2 | 2 | 3 |

Other dyes conventionally used for vital staining can also be used in the dye exclusion test. Non-electrolyte dyes are generally useful in such tests, e.g., eosin, Evans blue, and the like.

Larval Tolerance to Methanol (Solvent):

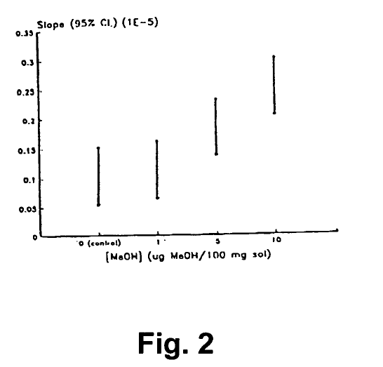

Active constituents of plants are typically extracted in organic solvents such as methanol. Thus, it is important to ensure that residual solvent does not adversely affect the larvae and in doing so influence the bioassay data. Studies of methanol toxicity for S. stercoralis indicate that a methanol concentration exceeding 5 μg/100 mL LNS (>50 times that detected in plant extracts using NMR analyses) is required to exert any significant effect on larval mortality. FIG. 2 shows a comparison of slopes relating to cumulative percentage mortality of S. stercoralis larvae in aqueous solutions containing methanol (Analysis of covariance: F=5.868, F_{0.05(3) 3,124}=2.67; p<0.05). Thus, residual solvent in methanol-extracted components does not affect bioassays using S. stercoralis.

Larval Tolerance to Most Commonly used Emulsifiers:

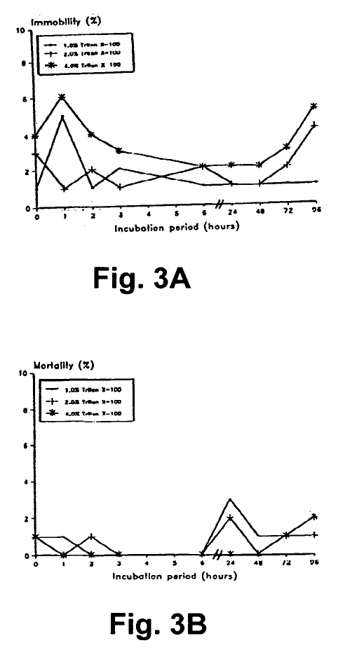

Triton® X-100 (octylphenoxy polyethoxyethanol) is recommended for use as an emulsifier, where necessary. A concentration of 1-4% in 0.01% ELNS had no effect on larval mobility or mortality. FIG. 3A shows the cumulative corrected percentage immobility, and FIG. 3B shows the cumulative corrected percentage mortality of S. stercoralis larvae by different concentrations of Triton® X-100.

A 1% solution of Triton® X-100 was found to disperse extracts of a variety of local plants dissolved in petroleum ether (PE), dicloromethane (DCM), or methanol-water (MW). However, Tween® 20 at a concentration of 0.1-0.4% in 0.1% ELNS appeared to be toxic to S. stercoralis larvae, and did not create a homogeneous and stable dispersion of globules of either petroleum ether- or dichloromethane-based extracts of a range of crude plant products tested. Other suitable surfactants for dispersing plant extracts can be developed by routine testing according to the described procedures.

Bioassay:

According to the invention larvae obtained by the procedure described above are contacted with materials that might exert a physiologic effect on them, and the effect of such materials on the larvae is evaluated. Typically, the materials to be evaluated are candidate anthelmintic compounds, which are evaluated for their effectiveness in immobilizing or killing the larvae.

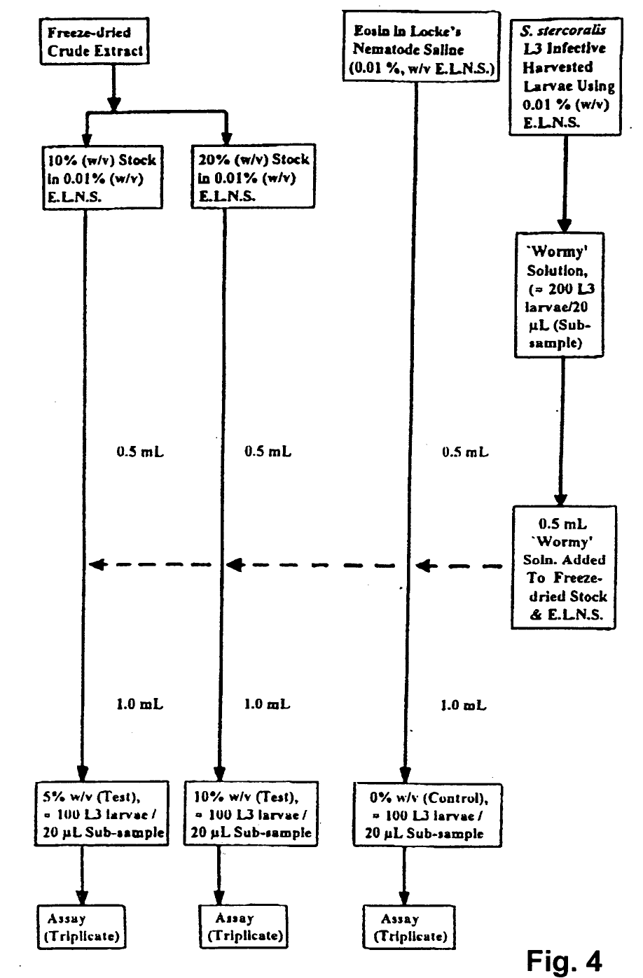

The flow chart of FIG. 4 illustrates a preferred example of the bioassay, in this instance, in determining the anthelmintic activity of a freeze-fried crude extract of a plant. At least three (3) replicates are preferably run in each assay, using >100 larvae per replicate. Whilst incubation times are selected by the investigator, the data should be subjected to standard probit analysis as an aid to interpretation. In the illustrated procedure, 1 mL of 0% (control), 10% and 20% w/v (weight per volume) extract stock solutions (0.1% ELNS) are diluted with 1 mL ELNS containing ˜200 larvae/20 μL, yielding 0% (control), 5% and 10% w/v extract containing ˜100 larvae/20 μL. Typically, 20 μL is a reasonable volume of ELNS to be placed on a large microscope slide for viability counts of ˜100 larvae.

The skilled practitioner will, of course, recognize that the above-described embodiments are illustrative of the present invention and not limiting.

1. A method for assessing a biological effect of a material on nematodes comprising:
maintaining an infection of Strongyloides stercoralis in a host animal;
harvesting larvae of said S. stercoralis from said host animal;
multiplying said larvae in vitro;
contacting said larvae with a material capable of having a biological effect on said larvae; and
observing said biological effect of said material on said larvae.

2. The method of claim 1, wherein said biological effect is toxicity toward said nematode.

3. The method of claim 2, wherein said biological effect is immobilization.

4. The method of claim 2, wherein said biological effect is lethality.

5. The method of claim 4, wherein said lethality is assessed by contacting said larvae with a vital staining dye.

6. The method of claim 5, wherein said vital staining dye is a non-electrolyte dye.

7. The method of claim 6, wherein said non-electrolyte dye is eosin.

8. The method of claim 4, wherein said material is a candidate anthelmintic material.

9. The method of claim 8, wherein said larvae are contacted with said candidate anthelmintic material in a medium containing said candidate anthelmintic material and said vital staining dye.

10. The method of claim 9, wherein said vital staining dye is eosin.

11. The method of claim 1, wherein said larvae are multiplied in coproculture.
